(19) United States
(12) Patent Application Publication
(10) Pub. No.: US 2021/0322691 A1
(43) Pub. Date: Oct. 21, 2021

(54) SYSTEMS AND METHODS FOR DETECTING RESPIRATORY MECHANICS

(71) Applicant: Covidien LP, Mansfield, MA (US)

(72) Inventors: Nancy F. Dong, Carlsbad, CA (US); Gabriel Sanchez, Valley Center, CA (US); Kun Li, San Diego, CA (US)

(73) Assignee: Covidien LP, Mansfield, MA (US)

(21) Appl. No.: 16/852,197

(22) Filed: Apr. 17, 2020

(51) Int. Cl. A61M 16/00; G16H 40/63

(52) U.S. Cl. CPC A61M 16/0003; A61M 16/024; A61M 2016/0027; A61M 2016/0042; G16H 40/63

(57) ABSTRACT

Systems and methods for detecting respiratory mechanics of a patient during mechanical ventilation. An example ventilator implemented method includes receiving pressure and flow measurements of breathing gas. Based on the measurements, the ventilator generates values for a derivative of an estimate of intrapleural pressure (Psync) of the patient, and the ventilator determines a rate of change of the generated Psync values over a portion of an expiratory phase of a breathing cycle. Based on the determined rate of change, the ventilator selects at least one of a triggering mode or a setting for ventilation, and the ventilator controls ventilation of the patient based on the selected triggering mode and/or the setting for ventilation.

### INTRODUCTION

Medical ventilator systems are used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different breath modes or settings have been created to provide better ventilation for patients in different scenarios, such as mandatory ventilation modes and spontaneous ventilation modes.

It is with respect to this general technical environment that aspects of the present technology disclosed herein have been contemplated. Although a general environment has been discussed, it should be understood that the examples described herein should not be limited to the general environment identified herein.

### SUMMARY

Aspects of the disclosure relate to providing novel systems and methods for detecting respiratory mechanics of a patient during mechanical ventilation. Based on the detected respiratory mechanics, the systems and method may control beginning (triggering) or ending (cycling) inspiration. The detected respiratory mechanics may also be used to adjust or select ventilation settings during mechanical ventilation.

In an aspect, the technology relates to a mechanical ventilator for providing mechanical ventilation of a patient. The ventilator includes, a pressure sensor, a flow sensor, a processor, and memory, storing instructions that when executed by the processor, cause the ventilator to perform a set of operations. The operations include receiving, from the pressure sensor, pressure measurements of breathing gas and receiving, from the flow sensor, flow measurements of the breathing gas. The operations also include, based on the pressure measurement and the flow measurement, generating a derivative of an estimate of intrapleural pressure (Psync signal) of the patient, determining a rate of change of the Psync signal over a portion of an expiratory phase, based on the determined rate of change, classifying respiratory mechanics as an obstructive respiratory condition, altering a cycling sensitivity based on the obstructive classification, and controlling ventilation of the patient based on the cycling sensitivity.

In an example, determining the rate of change of the Psync signal includes identifying a maximum value of the Psync signal during the expiratory phase; determining a Psync value for a threshold percentage of the identified maximum Psync value; and the determined rate of change is between the identified maximum Psync value and the determined Psync value for the threshold percentage. In another example, altering the cycling sensitivity comprises altering a percentage sensitivity value. In a further example, controlling ventilation of the patient includes cycling, from an inspiratory phase to an expiratory phase, based on the percentage sensitivity value. In yet another example, classifying the respiratory mechanics comprises comparing the determined rate of change to a mechanics threshold. In still another example the mechanics threshold is 30cmH₂O/s². In yet another example, the obstructive classification is based on the rate of change being below the mechanics threshold. In still yet another example, the operations further comprise, based on the determined rate of change, selecting a triggering mode. In another example, the selected triggering mode is one of flow triggering, pressure triggering, or signal distortion triggering.

In another aspect, the technology relates to a ventilator-implemented method for providing mechanical ventilation of a patient. The method includes receiving pressure measurements of breathing gas; receiving flow measurements of the breathing gas; based on the pressure measurement and the flow measurement, generating values for a derivative of an estimate of intrapleural pressure (Psync) of the patient; determining a rate of change of the generated Psync values over a portion of an expiratory phase of a breathing cycle; based on the determined rate of change, selecting at least one of a triggering mode or a setting for ventilation; and controlling ventilation of the patient based on the selected at least one of the triggering mode or the setting for ventilation.

In an example, determining the rate of change of the Psync values includes identifying a maximum of the Psync values during the expiratory phase; determining a Psync value for a threshold percentage of the identified maximum Psync value; and the determined rate of change is between the identified maximum Psync value and the determined Psync value for the threshold percentage. In another example, the method also includes based on the rate of change, classifying respiratory mechanics of the patient. In a further example, the respiratory mechanics of the patient is one of normal, restrictive, or obstructive. In yet another example, classifying the respiratory mechanics comprises comparing the determined rate of change to a mechanics threshold. In still another example, the mechanics threshold is 30 cmH₂O/s². In still yet another example, the patient is classified as obstructive if the rate of change is below the mechanics threshold.

In another aspect, the technology relates to a ventilator-implemented method for providing mechanical ventilation of a patient. The method includes receiving pressure measurements of breathing gas for at least a first breathing cycle and a second breathing cycle, the second breathing cycle occurring subsequent to the first breathing cycle; receiving flow measurements of the breathing gas for at least the first breathing cycle and a second breathing cycle; based on the pressure measurement and the flow measurement, generating values for a derivative of an estimate of intrapleural pressure (Psync) of the patient for at least the first breathing cycle and the second breathing cycle; for an expiratory phase of the first breathing cycle, determining a first rate of change of the generated Psync values; based on the determined first rate of change, selecting at least one of a first triggering mode or a first setting for ventilation; controlling ventilation of the patient during the second breathing cycle based on the selected at least one of the first triggering mode or the first setting; for an expiratory phase of the second breathing cycle, determining a second rate of change of the generated Psync values; based on the determined second rate of change, selecting at least one of a second triggering mode or a second setting for ventilation; and controlling ventilation of the patient during a third breathing cycle based on the selected at least one of the second triggering mode or the second setting for ventilation, wherein the third breathing cycle occurs after the second breathing cycle.

In an example, determining the first rate of change includes identifying a maximum of the Psync values during the expiratory phase of the first breathing cycle, determining a Psync value, in the expiratory phase of the first breathing cycle, for a threshold percentage of the identified maximum Psync value; and the determined first rate of change is between the identified maximum Psync value and the determined Psync value for the threshold percentage. In another example, the method further includes based on the second rate of change, classifying respiratory mechanics of the patient. In a further example, classifying the respiratory mechanics comprises comparing the determined rate of change to a mechanics threshold.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

The following drawing figures, which form a part of this application, are illustrative of aspects of systems and methods described below and are not meant to limit the scope of the invention in any manner, which scope shall be based on the claims.

FIG. 1 is a schematic diagram illustrating a ventilator with a respiratory mechanics detection system, in accordance with aspects of the disclosure.

FIG. 2 depicts a representation of an example algorithm that may be performed by the present technology.

FIG. 3 depicts a chart of a patient having restrictive respiratory disorders.

FIG. 4 depicts a chart of patient having normal respiratory mechanics.

FIG. 5 depicts a chart of a patient having obstructive respiratory disorders.

FIG. 6A depicts an example method for providing mechanical ventilation.

FIG. 6B depicts an example chart for a non-obstructive patient.

FIG. 6C depicts an example method for determining a rate of change of a signal.

FIG. 6D depicts an example method for classifying respiratory mechanics.

FIG. 7 depicts another example method for providing mechanical ventilation.

### DETAILED DESCRIPTION

Medical ventilators are used to provide a breathing gas to a patient who may otherwise be unable to breathe sufficiently. In modern medical facilities, pressurized air and oxygen sources are often available from wall outlets or pressurized tanks. Accordingly, ventilators may provide control valves (limiting or regulating pressure or flow) connected to sources of pressurized air and pressurized oxygen. The flow valves function to regulate flow so that respiratory gas having a desired concentration of oxygen is supplied to the patient at desired pressures and flow rates. Ventilators capable of operating independently of external sources of pressurized air are also available (such as ventilators with pumps, blowers, and/or fans).

As each patient may require a different ventilation strategy, modern ventilators can be customized for the particular needs of an individual patient. For example, several different ventilator modes, breath types, and/or settings have been created to provide clinically appropriate ventilation for patients in various different scenarios, such as mandatory ventilation modes and assist control ventilation modes. Assist control modes (also referred to herein as “spontaneous modes”) allow a spontaneously breathing patient to trigger inspiration during ventilation. In a spontaneous or assisted mode of ventilation, the ventilator begins (triggers) inspiration upon the detection of patient demand or patient effort to inhale. The ventilator ends inspiration and begins expiration (cycles to expiration) when a threshold is met or when a patient demand or effort for exhalation is detected.

The performance of a medical ventilator in responding to a patient effort to begin a new inspiration (trigger inhalation) or a patient effort to end an inspiration (cycle to exhalation) represents an important characteristic of a medical ventilator. A ventilator's inspiration trigger and expiration cycle response impact the patient's work of breathing and the overall patient-ventilator synchrony. The trigger and cycle responses of a ventilator are a function of a patient's inspiratory and expiratory behavior (breathing effort magnitude and timing characteristics), as well as the ventilator's gas delivery dynamics and flow control parameters (actuator response, delay, etc.).

Triggering delay time, cycling delay time, asynchrony index, metabolic work, pressure-time product, and other parameters are used to measure the patient-ventilator synchrony. The asynchrony index is the ratio between the number of asynchronous events and the total respiratory rate. Mis-triggering (“missing” a trigger by failing to provide inspiration in response to a patient demand to inhale) or delayed cycling (delayed inspiration termination in response to a patient demand to exhale) can increase the patient-ventilator asynchrony index. Similarly, auto-triggering (providing inhalation too early or delivering breath without patient effort) and premature cycling (terminating inspiration before patient's neural inspiration ends) can also increase the asynchrony index. Several different factors cause asynchrony events, such as variation in patient's breathing pattern, muscle strength, respiratory mechanics, ventilator performance, and ventilator characteristics.

In some triggering modes, a patient's inspiratory trigger is detected based on the magnitude of deviations (deviations generated by a patient's inspiratory effort) of a measured parameter from a determined baseline. For example, in flow triggering, the patient's inspiration effort is detected when the measured patient exhalation flow value drops below a flow baseline (i.e. the base flow) by a set amount (based on the triggering sensitivity). In pressure triggering, the patient's inspiration effort is detected when the measured expiratory pressure value drops below a pressure baseline (for example, the set PEEP value) by a set amount (based on triggering sensitivity). Another parameter that can be used for triggering is a derived signal, such as an estimate of the intrapleural pressure of the patient and/or the derivative of the estimate of the patient's intrapleural pressure. The term “intrapleural pressure,” as used herein, refers generally to the pressure exerted by the patient's diaphragm on the cavity in the thorax that contains the lungs, or the pleural cavity. The derivative of the intrapleural pressure value will be referred to herein as a “Psync” value that has units of pressure per time. An example of triggering and cycling based on the Psync value is provided in U.S. patent application Ser. No. 16/411,916 (“the '916 Application”), titled “Systems and Methods for Respiratory Effort Detection Utilizing Signal Distortion” and filed on May 14, 2019, which is incorporated herein by reference in its entirety. That triggering mode discussed in the '916 Application is referred to herein as the “signal distortion” triggering mode. As discussed in the '916 Application, the signal distortion triggering mode may operate on the Psync signal or other signals, such as flow or pressure.

Each type of triggering mode (e.g., flow triggering, pressure triggering, signal distortion triggering, etc.) has different benefits and drawbacks for different types of patients. In addition, various ventilation settings may be adjusted to better suit each type of patient. By selecting the best-suited triggering mode and the best-suited settings within that triggering mode, patient synchrony may be improved, resulting in a decrease in patient discomfort. Identifying the proper triggering mode and settings, however, continue to be a challenge.

The present technology provides for additional solutions and improvements for identifying and selecting triggering modes and settings for ventilation. The present technology analyzes the measured and/or derived respiratory parameters (flow, pressure, Psync, etc.) to determine or detect respiratory mechanics of the patient receiving the mechanical ventilation. For instance, the technology may detect that the patient has flow-limited breathing, such as is common with obstructive respiratory disorders (e.g., air trapping, flow limitations, chronic obstructive pulmonary disease (COPD)). The technology may also detect whether the patient has other breathing conditions, such as restrictive respiratory disorders (e.g., acute respiratory distress syndrome (ARDS)), or if the respiratory mechanics of the patient are clinically normal for purposes of ventilation. Based on the detected respiratory mechanics of the patient, a triggering mode or a combination of triggering modes may be initiated. In addition, ventilation settings, such as settings within the triggering mode, may also be selected based on the detected respiratory mechanics. Ventilation may then be controlled based on the selected triggering mode(s) and setting(s).

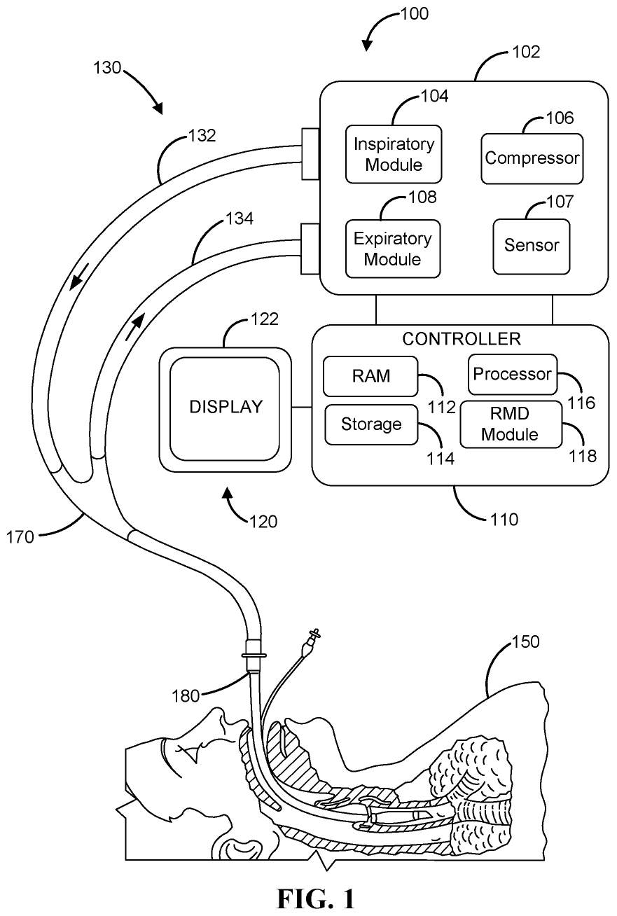

FIG. 1 illustrates a schematic diagram of an aspect of an exemplary ventilator 100 connected to a human patient 150. Ventilator 100 includes a respiratory mechanics detection (RMD) module 118. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown, or other airway tubes such as tracheostomy tubes) or a non-invasive (e.g., nasal mask or prongs) patient interface 180. Pneumatic system 102 includes an expiratory module 108 and an inspiratory module 104. The ventilator 100 also includes one or more sensors 107 such as pressure, flow, temperature, and other sensors communicatively coupled to the ventilator.

In an embodiment, the sensors 107 are non-invasive to the patient. In an embodiment, the non-invasive sensors 107 are non-contact, meaning they do not physically touch the patient. In an embodiment, the sensors 107 are located within the mechanical ventilator 100. Sensors are referred to as non-invasive when the sensors are located externally to patient. For example, sensors located in the patient wye 170, in the expiratory module 108, in the inspiratory module 104, or on the patient's finger are all external to the patient and are non-invasive. Sensors are referred to herein as invasive when the sensors are located within the patient or placed inside the patient's body, such as sensors located in an endotracheal tube, near a patient diaphragm, or on an esophageal balloon. While invasive sensors can provide great patient data or measurements, these sensors can often be hard to maintain or keep properly positioned. In an embodiment, the respiratory mechanics detection operations and other actions on the ventilator are accomplished with non-invasive and/or non-contact sensors, and without adding any additional sensors to the ventilator 100.

In some examples, the RMD module 118 monitors the parameter signal at each sample period. The sample period as used herein refers to a discrete period of time used to monitor a physiological parameter. In some aspects, the sample period is a computation cycle for the ventilator 100. In some aspects, the sample period is every 1 milliseconds (ms), 2 ms, 3 ms, 4 ms, 5 ms, 10 ms, 15 ms, 20 ms, 25 ms, 30 ms, 50 ms, 100 ms, or other similar period. This list is exemplary only and is not meant to be limiting. Any suitable sample period for monitoring a physiological parameter of the patient may be utilized by the ventilator 100 as would be understood by a person of skill in the art. In an example, the RMD module 118 receives a sensor output (such as a raw or filtered measurement from a sensor 107), determines the physiological parameter from the sensor output (such as calculating a flow waveform from a flow sensor measurement), and provides the physiological parameter to other components of the ventilator 100 (such as pneumatic system 102, expiratory module 108, inspiratory module 104, processor 116, or controller 110). Alternatively or in conjunction, the RMD module 118 receives the calculated physiological parameter (such as a flow waveform) calculated elsewhere in the system (such as pneumatic system 102, expiratory module 108, inspiratory module 104, processor 116, or controller 110).

Ventilation tubing system 130 (or patient circuit 130) may be a two-limb as shown (or a one-limb) circuit for carrying gases to and from (or only to) the patient 150. In a two-limb aspect, a fitting, typically referred to as a “wye-fitting” 170, may be provided to couple the patient interface 180 (shown as an endotracheal tube in FIG. 1) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106, accumulator and/or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 controls an inspiratory valve to deliver gases to the patient 150 through the inspiratory limb 132 according to prescribed ventilatory settings and modes, such as mandatory, spontaneous, and/or assist modes. The expiratory module 108 controls an expiratory valve to release gases from the patient's lungs according to prescribed ventilatory settings and modes, such as mandatory, spontaneous, and/or assist modes.

The sensors 107 may be located in the pneumatic system 102, in an accumulator, in or affixed to ventilation tubing system 130 or the wye 170, in components or modules of the ventilator 100, and/or on the patient 150. For example, sensors 107 may be coupled to the inspiratory module 104 and/or expiratory module 108 for detecting changes in, for example, circuit pressure and/or flow. FIG. 1 illustrates a sensor 107 (e.g., flow sensor, pressure sensor, etc.) in pneumatic system 102. Sensors 107 may generate output, such as measurements, and send this output to (and communicate with) various components of ventilator 100, e.g., pneumatic system 102, other sensors 107, expiratory module 108, inspiratory module 104, processor 116, controller 110, RMD module 118, and any other suitable components and/or modules. For example, in some aspects, the one or more sensors 107 of the ventilator 100 include an inspiratory flow sensor and an expiratory flow sensor. Ventilatory parameters may be directly monitored by one or more sensors 107, as described above, or may be indirectly monitored or estimated by derivation according to the Equation of Motion or other known relationships from the monitored parameters.

The pneumatic system 102 may include a variety of other components, including mixing modules, valves, tubing, accumulators, filters, etc. Controller 110 is operatively coupled with pneumatic system 102, signal measurement and acquisition systems (e.g., sensor(s) 107), and an operator interface 120 that may enable an operator to interact with the ventilator 100 (e.g., change ventilator settings, select operational modes, view monitored parameters, etc.). In some aspects, the operator interface 120 of the ventilator 100 includes a display 122 communicatively coupled to ventilator 100. Display 122 may provide various input screens, for receiving clinician input, and various display screens, for presenting useful information to the clinician. In aspects, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122.

Display 122 may also provide useful information in the form of various ventilatory data regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100. In some aspects, the display 122 illustrates a physiological parameter, a graph or waveform of the physiological parameter, a detected patient trigger, a trigger sensitivity, the detected respiratory mechanics, and/or any other information known, received, or stored by the ventilator 100.

In some aspects, controller 110 includes memory 112, one or more processors 116, storage 114, and/or other components of the type commonly found in command and control computing devices. Controller 110 may further include the RMD module 118 as illustrated in FIG. 1. In alternative aspects, the RMD module 118 is located in other components of the ventilator 100, such as in the pressure generating system 102 (also known as the pneumatic system 102) or inspiratory module 104.

The memory 112 includes non-transitory, computer-readable storage media that stores and/or encodes software (or computer readable instructions) that is executed by the processor 116 and which controls the operation of the ventilator 100. In an aspect, the memory 112 includes one or more solid-state storage devices such as flash memory chips. In an alternative aspect, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

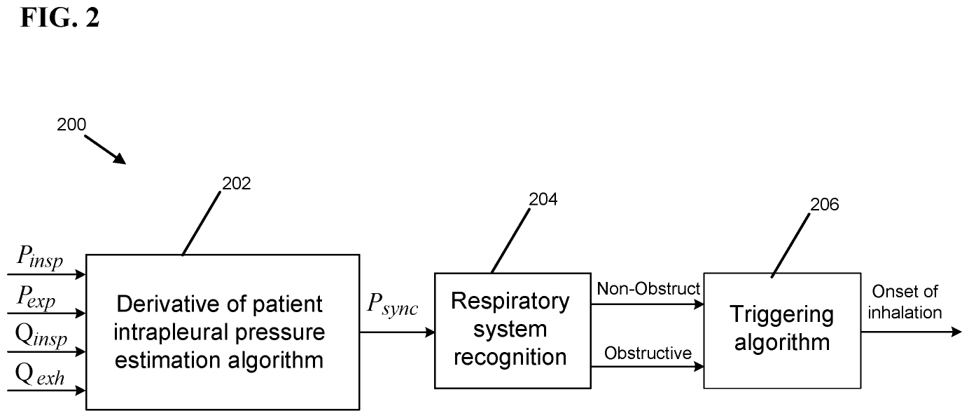

FIG. 2 depicts a representation of an example algorithm 200 that may be performed by the present technology. The example algorithm 200 may be performed by the ventilator, and may more specifically be performed by the RMD module of the ventilator. At a first stage 202 of the algorithm, a derivative of an estimated intrapleural pressure for the patient, i.e., Psync, is generated. The first stage 202 of the algorithm receives as input at least one pressure parameter and one flow parameter. In the example depicted, the inputs include an inspiratory pressure (Pᵢₙₛₚ) value, an expiratory pressure (Pₑₓₚ) value, an inspiratory flow (Qᵢₙₛₚ) value, an expiratory flow (Qₑₓₚ) value. The Pᵢₙₛₚ value may be an inspiratory pressure transducer measurement, the Pₑₓₚ value may be an expiratory pressure transducer measurement, the Qᵢₙₛₚ value may be an inspiratory flow sensor measurement, and the Qₑₓₚ value may be an expiratory flow sensor measurement. The pressure and flow values may be generated or derived from the various sensors of the ventilator, such as sensors 107 depicted in FIG. 1 and discussed above. The flow and pressure values may be received as one or more signals that are substantially continuous. For example, the flow and pressure values may be measured and/or recorded for every sampling period (e.g., every 5 ms).

The flow and pressure values may thus be provided as one or more vectors, arrays of values, and/or substantially continuous signals. From the input values, the first stage 202 of the algorithm 200 generates a Psync value or a Psync signal. For example, the Psync signal may be a series of Psync values for each of the sampling periods for which the input values were provided. An example algorithm for determining and estimate of intrapleural pressure is described in U.S. patent application Ser. No. 12/980,583, filed Dec. 29, 2010, which is incorporated herein by reference in its entirety. Additional or alternative methods may be used for generating an estimate of intrapleural pressure and a derivative thereof.

The second stage 204 of the algorithm 200 receives the Psync signal as input. The second stage 204 analyzes the Psync signal to determine or detect respiratory mechanics of the patient receiving the mechanical ventilation. For example, the rate of change or slope of the Psync signal is determined, as discussed in further detail below. In other examples, the rate of change may be based on a flow signal. Based on the determined slope or rate of change, respiratory mechanics of the patient are determined. For instance, a determination as to whether the respiratory mechanics of the patient are normal, indicative of a restrictive respiratory disorder (e.g., ARDS), or indicative of an obstructive respiratory disorder (e.g., COPD) may be made in the second stage 204 of the algorithm 200. The determination of the respiratory mechanics is then output to a third stage 206 of the algorithm 200.

The third stage 206 of the algorithm 200 uses the determined respiratory mechanics to select, adjust, or control breath delivery modes and settings on the ventilator. For example, based on the detected respiratory mechanics, the ventilator may automatically select (or prompt a user to select) a triggering mode, such as a signal distortion triggering mode, a flow triggering mode, or a pressure triggering mode. In some examples, a combination of triggering modes may be utilized. Further, the ventilator may adjust (or prompt a user to adjust) the settings of the selected triggering mode. Settings may include sensitivity settings, among other settings. Ventilation is then controlled based on the selected triggering mode and settings determined in the third stage 206 of the algorithm 200. In addition to triggering mode and triggering settings, other breath delivery modes or settings may be determined in the third stage 206 of the algorithm, such as cycling mode or cycling settings, among others.

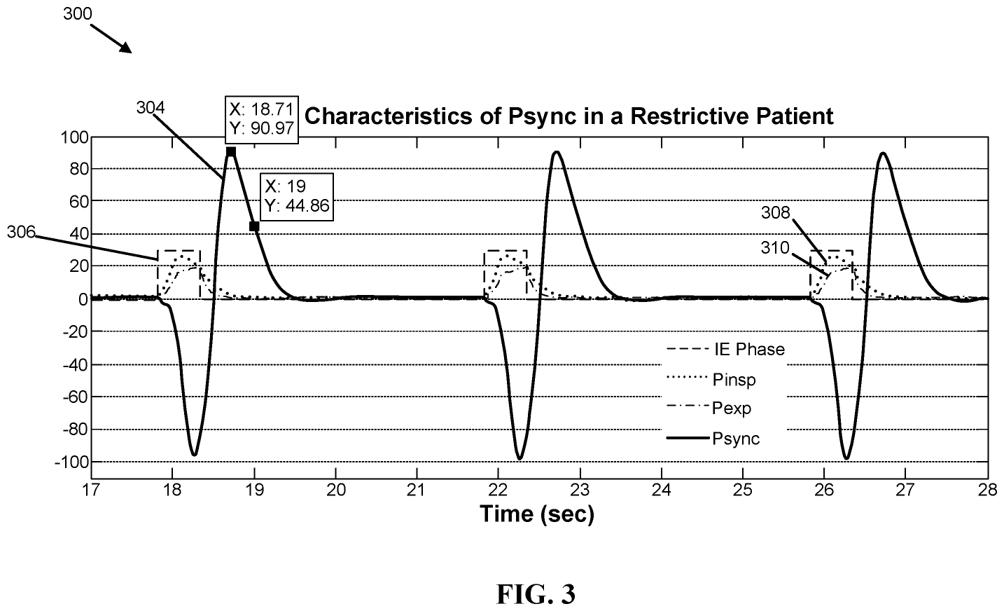

FIGS. 3-5 depict Psync and pressure signals indicative of different respiratory mechanics. In each of the charts in FIGS. 3-5, the ventilator settings remained the same, but the plotted signals changed due to different characteristics of the respective patients. In an embodiment, the characteristics of the signals, such as morphology, shape, slope, noise, and other metrics, are used to determine the respiratory mechanics of the patient.

More specifically, FIG. 3 depicts a chart 300 of respiratory parameters from a patient having restrictive lung condition. The chart 300 includes a plot of a Psync signal 304. The inspiratory pressure 308, expiratory pressure 310, and the breath phase 306 (i.e., the IE phase) are also plotted. The IE phase 306 is depicted as a square waveform, which changes between a low level and a high level. When the IE phase 306 is high, the ventilator is in an inspiratory phase, and when the IE phase 306 is low, the ventilator is in an expiratory phase. The units of the y-axis are in cmH₂O for the pressure signals 308, 310 and cmH₂O/s for the Psync signal 304. The IE phase is unitless, and simply indicates whether the ventilator is in an inspiratory phase or an expiratory phase.

To determine the respiratory mechanics of the patient for which the chart 300 was generated, the ventilator analyzes a rate of change or slope of the Psync signal during an expiratory phase of a breathing cycle. In an example, the rate of change is analyzed in a selected portion of the Psync waveform between a peak or maximum of the Psync signal and a threshold percentage of that peak. The threshold percentage may be a particular percentage value, such as 50% of the Psync peak. In other examples, the percentage threshold may be between 30-80%, such as 30%, 40%, 50%, 60%, 70%, 80%, or any value within that range. The maximum or peak Psync value may be referred to as P_{sync_peak}, the threshold percentage Psync value may be referred to as P_{sync_% thresh}, and the rate of change or slope of the Psync signal may be referred to as P_{sync_slope}. Accordingly, determining the rate of change or slope between the maximum Psync value (P_{sync_peak}) and the Psync signal at the threshold percentage (P_{sync_% thresh}) may be determined by the following Equation #1:

$${P}_{sync\_slope} = \frac{{P}_{sync\_peak} - {P}_{sync\_ \% thresh}}{duration} , \qquad ( Eq . 1 )$$

where duration is the amount of time that passed between the Psync signal dropping from P_{sync_peak} to P_{sync_% thresh}. In examples where triggering occurs before the Psync signal drops to the P_{sync_% thresh}, the final Psync value measured during the expiratory phase may be used in place of the P_{sync_% thresh} value.

In the chart 300, the maximum Psync signal value (P_{sync_peak}) for the first exhalation phase depicted is 90.97 cmH₂O/s and occurs at 18.71 seconds. For the chart 300, the threshold percentage of 50% was utilized. Accordingly, the first Psync data which has value of lower than or equal to 50% of the P_{sync_peak} is 44.86 cmH₂O/s. In chart 300, that value occurs at 19 seconds. Thus, the P_{sync_peak} has (x, y) coordinates of (18.71, 90.97) and the P_{sync_% thresh} has (x, y) coordinates of (19, 44.86). The rate of change between those values is thus 159 cmH₂O/s², which can be seen by inserting those values into Equation 1, as shown below:

$${P}_{sync\_slope} = \frac{90.97 - 44.86}{19 - 18.71} = 159 {cmH}_{2} 0 / {s}^{2}$$

To determine the respiratory mechanics of the patient, the P_{sync_slope} may then be compared to a mechanics threshold or a set of mechanics thresholds. An example set of two mechanics thresholds that may be used to categorize patients is provided in Table 1 below:

TABLE 1

| Respiratory Mechanics | Mechanics Threshold |
|---|---|
| Obstructive (e.g., COPD) | P_{sync}_{_{—}}ₛₗₒₚₑ < MT₁ |
| Normal | MT₁ ≤ P_{sync}_{_{—}}ₛₗₒₚₑ ≤ MT₂ |
| Restrictive (e.g., ARDS) | P_{sync}_{_{—}}ₛₗₒₚₑ > MT₂ |

As indicated in Table 1, if the P_{sync_slope} value is less than a first respiratory mechanics threshold (MT₁), then the patient may be classified as having an obstructive condition, such as COPD. If the P_{sync_slope} value is greater than or equal to a first respiratory mechanics threshold (MT₁) but less than or equal to a second, higher mechanics threshold (MT₂), then the patient may be classified as normal. If the P_{sync_slope} value is greater than the second mechanics threshold (MT₂), then the patient may be classified as having a restrictive condition, such as ARDS. In some examples, the first mechanics threshold may be within a range of 20-50 cmH₂O/s², such as 30 cmH₂O/s². The second mechanics threshold may be within a range of 90-130 cmH₂O/s², such as 100 cmH₂O/s². In such an example, a P_{sync_slope} of 159 cmH₂O/s², such as slope determined for chart 300, is greater than MT₂ and leads to a classification of restrictive.

In other examples, more or fewer than two mechanics thresholds may be utilized to make more or fewer classifications of patients. For instance, a single mechanics threshold may be used to classify a patient as having an obstructive condition (e.g., COPD) or a non-obstructive condition (e.g., normal or ARDS). An example of using a single mechanics threshold for such a determination is provided in Table 2 below:

TABLE 2

| Respiratory Mechanics | Mechanics Threshold |
|---|---|
| Obstructive | P_{sync}_{_{—}}ₛₗₒₚₑ < MT |
| Non-Obstructive | MT ≤ P_{sync}_{_{—}}ₛₗₒₚₑ |

As indicated in Table 2, if the P_{sync_slope} value is less than a respiratory mechanics threshold (MT), then the patient may be classified as obstructive. If the P_{sync_slope} value is greater than or equal to the first respiratory mechanics threshold (MT), then the patient may be classified as non-obstructive. The mechanics threshold in such an example may be within a range of 20-50 cmH₂O/s², such as 30 cmH₂O/s². In such an example, a P_{sync_slope} of 159 cmH₂O/s², as shown in chart 300, is greater than MT and leads to a classification of non-obstructive.

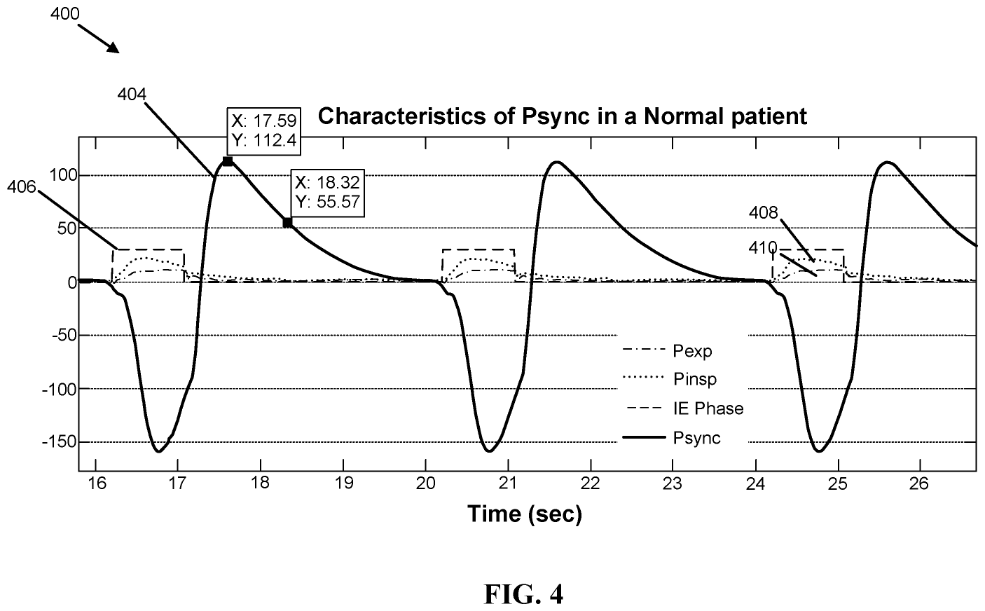

FIG. 4 depicts a chart 400 of respiratory parameters from a patient having normal respiratory mechanics. The chart 400 in FIG. 4 is similar to the chart 300 in FIG. 3 with the exception that the chart 400 shows parameters for a patient having normal respiratory mechanics, rather than a patient with a restrictive lung condition. For instance, chart 400 includes a plot of a Psync signal 404. The inspiratory pressure 408, expiratory pressure 410, and the breath phase 406 (i.e., the IE phase) are also plotted. As can be seen from a comparison of the chart 400 in FIG. 4 and the chart 300 in FIG. 3, the slope of the Psync signal in the expiratory phase in chart 400 is less steep than the slope of the Psync signal in the expiratory phase in chart 300. The difference in slope is due to the difference of respiratory mechanics of the patients.

The slope of the Psync signal (P_{sync_slope}) in chart 400 may be determined in the same manner as described above with respect to chart 300, such as by using Equation 1. In the chart 400, the maximum of peak of the Psync signal (P_{sync_peak}) is 112.4 cmH₂O/s and occurs at 17.59 seconds. Using the percentage threshold of 50%, the P_{sync_% thresh} is 55.57 cmH₂O/s and occurs at 18.32 seconds. Thus, the P_{sync_slope} is 77.85 cmH₂O/s². The calculation of P_{sync_slope} is provided below by inserting the determined values into Equation 1:

$${P}_{sync\_slope} = \frac{112.4 - 55.57}{18.32 - 17.59} = 77.85 {cmH}_{2} 0 / {s}^{2}$$

Based on that determined P_{sync_slope} value of 77.85 cmH₂O/s², the patient's respiratory mechanics may be classified. For instance, the patient's respiratory mechanics may be classified using two mechanics thresholds (MT₁, MT₂) as discussed above with reference to Table 1. Using an example where the first mechanics threshold (MT₁) is 30 cmH₂O/s² and the second mechanics threshold (MT₂) is 100 cmH₂O/s², the P_{sync_slope} value of 77.85 cmH₂O/s² is between MT₁ and MT₂ and causes the patient's respiratory mechanics to be classified as normal. As another example, the patient's respiratory mechanics may be classified using a single mechanics threshold (MT) as discussed above with reference to Table 2. Using an example where the mechanics threshold (MT) is 30 cmH₂O/s², the P_{sync_slope} value of 77.85 cmH₂O/s² is greater than MT and causes the patient's respiratory mechanics to be classified as non-obstructive.

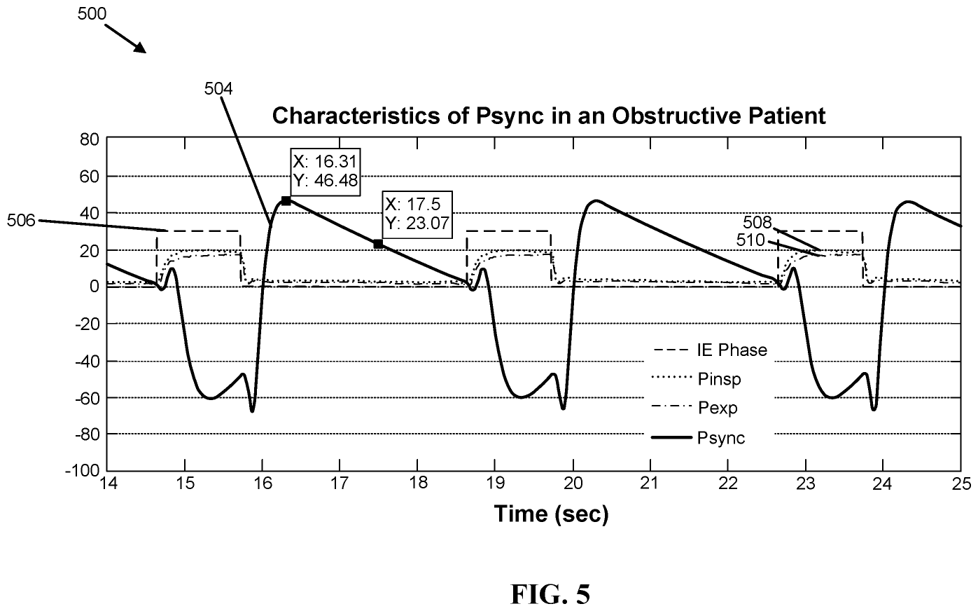

FIG. 5 depicts a chart 500 of respiratory parameters from a patient having obstructive lung condition. The chart 500 in FIG. 5 is similar to the charts 300, 400 in FIGS. 3-4 with the exception that the chart 500 is for a patient having obstructive lung condition. For instance, chart 500 includes a plot of a Psync signal 504. The inspiratory pressure 508, expiratory pressure 510, and the breath phase 506 (i.e., the IE phase) are also plotted. As can be seen from a comparison of the chart 500 in FIG. 5 and the charts 300, 400 in FIGS. 3-4, the slope of the Psync signal in the expiratory phase in chart 500 is less steep than the slopes of the Psync signals in the expiratory phase in charts 300, 400. The difference in slope is due to the difference of respiratory mechanics of the patients.

The slope of the Psync signal (P_{sync_slope}) in chart 500 may be determined in the same manner as described above with respect to charts 300, 400, such as by using Equation 1. In the chart 500, the maximum of peak of the Psync signal (P_{sync_peak}) is 46.48 cmH₂O/s and occurs at 16.31 seconds. Using the percentage threshold of 50%, the P_{sync_% thresh} is 23.07 cmH₂O/s and occurs at 17.5 seconds. Thus, the P_{sync_slope} is 19.67 cmH₂O/s². The calculation of P_{sync_slope} is provided below by inserting the determined values into Equation 1:

$${P}_{sync\_slope} = \frac{46.48 - 23.07}{17.5 - 16.31} = 19.67 {cmH}_{2} 0 / {s}^{2}$$

Based on that determined P_{sync_slope} value of 19.67 cmH₂O/s², the patient's respiratory mechanics may be classified. For instance, the patient's respiratory mechanics may be classified using two mechanics thresholds (MT₁, MT₂) as discussed above with reference to Table 1. Using an example where the first mechanics threshold (MT₁) is 30 cmH₂O/s² and the second mechanics threshold (MT₂) is 100 cmH₂O/s², the P_{sync_slope} value of 19.67 cmH₂O/s² is less than MT₁ and causes the patient's respiratory mechanics to be classified as obstructive. As another example, the patient's respiratory mechanics may be classified using a single mechanics threshold (MT) as discussed above with reference to Table 2. Using an example where the mechanics threshold (MT) is 30 cmH₂O/s², the P_{sync_slope} value of 19.67 cmH₂O/s² is less than MT and causes the patient's respiratory mechanics to be classified as obstructive.

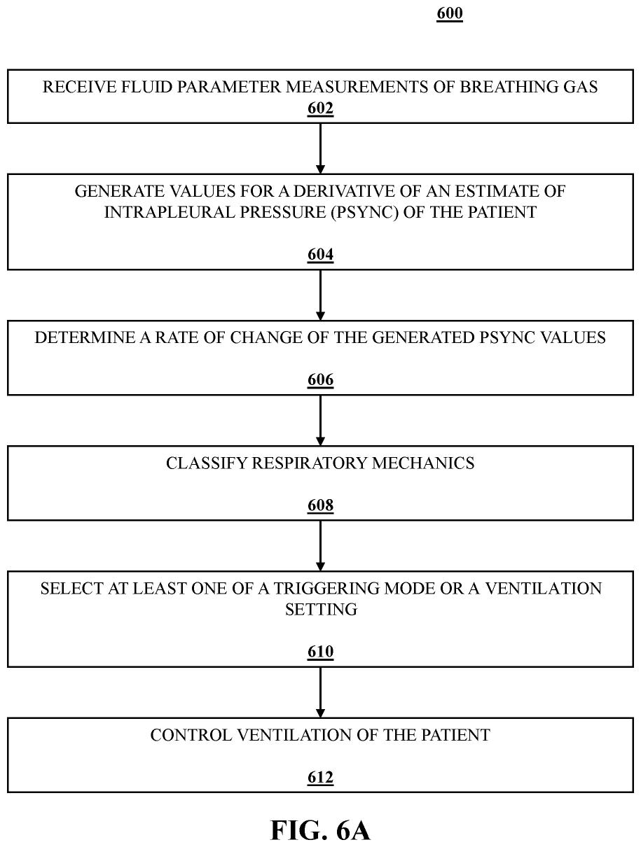

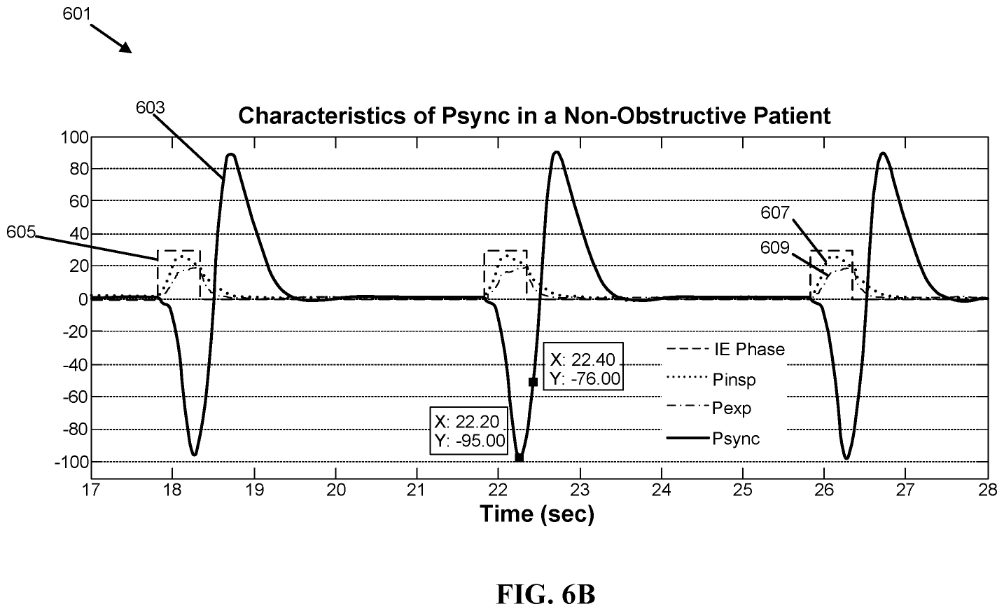

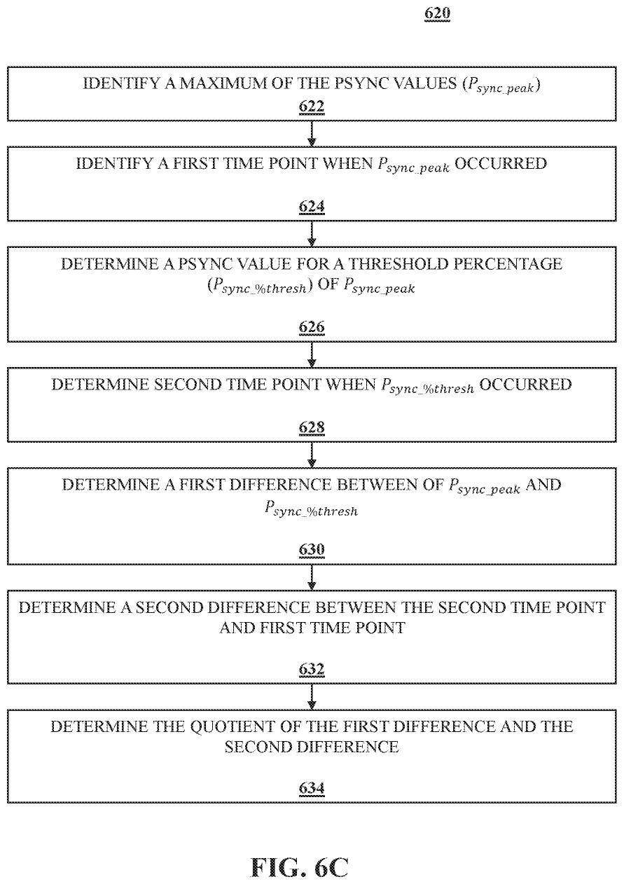

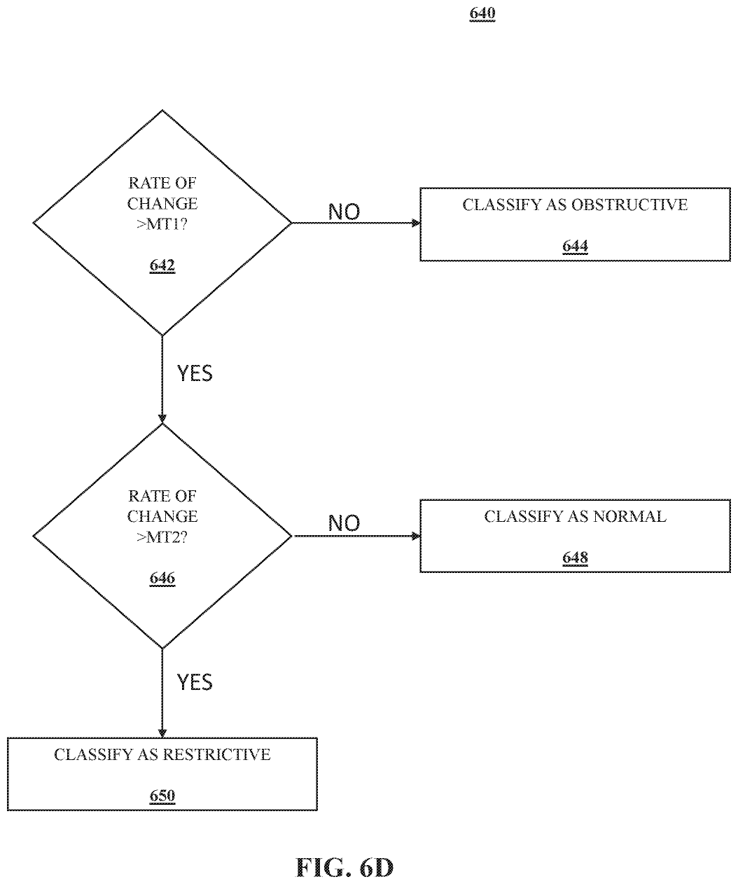

FIG. 6A depicts an example method 600 for providing mechanical ventilation based on detected respiratory mechanics. Method 600 may be performed by the ventilator, and/or more specifically, may be performed by a computing device within the ventilator. At operation 602, fluid parameter measurements are received or otherwise obtained. For example, pressure measurements of a breathing gas provided by the ventilator may be received. The pressure measurements may include a Pᵢₙₛₚ value that is an inspiratory pressure transducer measurement and a Pₑₓₚ value that is an expiratory pressure transducer measurement. Flow measurements of the breathing gas may also be received. The flow measurements may include a Qᵢₙₛₚ value that is an inspiratory flow sensor measurement and a Qₑₓₚ value that is an expiratory flow sensor measurement. The pressure measurements and flow measurements may be received for each sample period or computation cycle of the ventilator. Additional parameters may also be received in operation 602.

At operation 604, values for a derivative of an estimate of intrapleural pressure (Psync) are generated based on the measurements received in operation 602. Generating the Psync values may be performed as described herein and/or as set forth in U.S. patent application Ser. No. 12/980,583, filed Dec. 29, 2010, which is incorporated herein by reference in its entirety. The values for the Psync may form or be part of a Psync signal representing the derivative of the estimate of intrapleural pressure. At operation 606, a rate of change of the Psync values during an expiratory phase of a breathing cycle is determined. The rate of change of the Psync values may be determined as described herein, such as by using Equation 1 provided above.

Based on the rate of change determined in operation 606, the respiratory mechanics of the patient may be classified in operation 608. For example, the respiratory mechanics of the patient may be classified based on comparing the rate of change determined in operation 606 to one or more mechanics thresholds (MTs), as described above. Based on that comparison, the respiratory mechanics of the patient may be classified as restrictive (e.g., ARDS), normal, or obstructive (e.g., COPD). In other examples, the respiratory mechanics of the patient may be classified as obstructive or non-obstructive.

At operation 610, based on the determined rate of change in operation 606 and/or the classification of the respiratory mechanics in operation 608, at least one a triggering mode and/or a setting for ventilation is determined. The triggering mode may be selected from one of a flow triggering mode, a pressure triggering mode, or a signal distortion triggering mode, among other types of triggering modes.

Some triggering modes may be more appropriate for different patients based on the respective respiratory mechanics of the patient. For example, the signal distortion triggering mode based on the Psync signal may be more appropriate for patients that have obstructive respiratory mechanics, such as patients having COPD. However, more traditional pressure or flow triggering modes may be more appropriate for patients that are classified as normal or restrictive, such as patients having ARDS. For instance, in some cases, the use of the signal distortion triggering mode based on Psync in normal or ARDS patients may result in a delay in triggering as compared to a flow triggering method. In testing performed for an ARDS patient, the use of flow triggering (based on a flow signal) was found to trigger a new inhalation on average 100 ms earlier than the use of signal distortion triggering (based on the Psync signal). In contrast, in testing performed on a COPD patient, substantially more (roughly 50%) patient triggering efforts were detected by using the signal distortion triggering mode based on Psync rather than a flow triggering (based on a flow signal), even with a low flow triggering sensitivity of 1.0 liters per minute (lpm). Thus, for a patient classified as obstructive in operation 608, a signal distortion triggering mode may be selected at operation 610, whereas for a patient classified as restrictive or ARDS in operation 608, a pressure or flow triggering mode may be selected in operation 610.

Operation 610 may also, or alternatively, include selecting or altering a setting of ventilation, such as a setting of a triggering mode or cycling mode. For example, triggering modes and cycling modes generally have sensitivity settings. The sensitivity related to triggering inspiration may be referred to as “Isens” and the sensitivity related to cycling from inspiration to expiration may be referred to as “Esens.” The sensitivity setting controls how quickly the ventilator triggers a new inhalation (trigger sensitivity, such as Isens) or cycles to a new exhalation (cycle sensitivity, or Esens). A higher sensitivity causes the ventilator to trigger or cycle more quickly, and a lower sensitivity causes the ventilator to trigger or cycle less quickly. The sensitivity settings may be initially set by a clinician or set automatically by the ventilator based on the classification of the patient's respiratory mechanics in operation 608 or the rate of change determined in operation 606. In an example, the settings for Isens and Esens range from −2 to +2, and each setting corresponds to a sensitivity value. Accordingly, each sensitivity setting may be mapped to a sensitivity value, which may be a percentage as discussed below.

In operation 610, the settings may change based on the classification of the patient's respiratory mechanics in operation 608 or the rate of change determined in operation 606. In an example of an alteration of the Esens setting, the setting itself may remain the same, but the corresponding sensitivity value that is mapped to each Esens setting may change. For instance, a clinician may select a particular Esens setting between −2 to +2. The selected Esens setting is mapped to a sensitivity value, and that sensitivity value may change based on the classification of the patient's respiratory mechanics. As an example, a set of Esens settings and the corresponding sensitivity or threshold values based on the determined respiratory mechanics are provided below in Table 3:

TABLE 3

| | |
|---|---|
| Esens | Sensitivity Values |

| Setting | Obstructive | Non-obstructive |
|---|---|---|
| −2 | 95% | 90% |
| −1 | 90% | 80% |
| 0 | 80% | 65% |
| +1 | 60% | 40% |
| +2 | 30% | 10% |

As shown in the table, the sensitivity values that are utilized may be adjusted based on the determined respiratory mechanics. In the example depicted in Table 3, each Esens setting (e.g., −2 to +2) has a corresponding sensitivity value based on the type of cycling mode that is selected. The example values provided in Table 3 may be for a cycling mode that causes the ventilator to cycle to an expiratory phase when the Psync signal reaches a certain percentage of a minimum value (e.g., the bottom of a valley of the signal or negative peak) of the Psync signal for the current inspiratory phase. The certain percentage may be determined from Table 3. For example, if the Esens value has been set to −1 and the respiratory mechanics of the patient are determined to be obstructive, cycling will occur when the Psync signal reaches 90% of the minimum Psync value for the current inspiratory phase. Accordingly, the sensitivity value may be automatically changed by the ventilator based on the determined respiratory mechanics while the Esens setting (e.g., −2 to +2) remains the same. Thus, the mapping of the Esens settings to the sensitivity values based on respiratory mechanics allows for a shift of the entire range of Esens settings to become more or less sensitive. In addition, once the sensitivity value has been mapped, the full range of Esens settings are still available for selection by a clinician. For example, at any point, a clinician may still change the Esens setting to a different setting, such as a setting between −2 and +2.

FIG. 6B depicts an example chart 601 for a non-obstructive patient to provide an illustration of cycling based on an Esens setting. The example chart 601 is similar to the charts depicted in FIGS. 3-5. For instance, example chart 601 a plot of a Psync signal 603. The inspiratory pressure 607, expiratory pressure 609, and the breath phase 605 (i.e., the IE phase) are also plotted. For purposes of this example, assume that the medical professional has set the Esens setting to −1. Thus, based on Table 3 above, the sensitivity value for this example is 80%. In the chart 601, the minimum Psync value for the second breathing cycle is −95 cmH₂O/s and is found at the bottom of the plotted negative valley. Based on the sensitivity value, the threshold value for cycling is therefore −76 cmH₂O/s (i.e., 80% of 95 cmH₂O/s). Accordingly, in such an example, when the Psync value crosses that threshold of −76 cmH₂O/s and is trending upward, the ventilator cycles from the inspiratory phase to an expiratory phase.

Returning to FIG. 6A, other settings outside of the sensitivity settings may also be altered in operation 610. For instance, for a signal distortion triggering mode, the type of signal that is analyzed may be selected in operation 610. As an example, the analyzed signal may be a Psync signal, a flow signal, or a combination of the two. Accordingly, in operation 610, the ventilator selects a Psync signal, a flow signal, or both based on the determined respiratory mechanics of the patient. For example, as discussed above, a flow signal may be better for restrictive respiratory mechanics and a Psync signal may be better for obstructive respiratory mechanics. The signal distortion triggering analysis is then performed on the selected signal(s). For example, if both the flow signal and the Psync signal are selected, triggering may occur when the signal distortion criteria are first met for either the flow signal or the Psync signal.

At operation 612, ventilation of the patient is controlled based on the triggering mode and/or the ventilation setting(s) that were selected in operation 610. For example, if a signal distortion triggering mode was selected in operation 610, triggering of an inspiratory phase for the following breathing cycle is performed based on signal distortion criteria. In some examples, method 600 may be performed on a breath-by-breath basis. That is, the respiratory rate of change of the Psync signal and/or the respiratory mechanics of the patient is determined in every breathing cycle. Thus, the triggering modes and/or the settings may potentially change in every breathing cycle if the respiratory mechanics of the patient change. In other examples, the rate of change of the Psync signal and/or the respiratory mechanics of the patient may be determined in different intervals, such as once every five or ten breaths, among other possible intervals. In yet other examples, the rate of rate of change of the Psync signal and/or the respiratory mechanics of the patient may be determined for a first breathing cycle, or a first set of breathing cycles, and then the triggering mode and/or settings maybe used for all or a majority of breathing cycles during ventilation of the patient until the patient is removed from the ventilator.

FIG. 6C depicts an example method 620 for determining a rate of change of a signal. The method 620 in FIG. 6C may be used in operation 608 of method 600 depicted in FIG. 6A. In operation 622 of method 620, a maximum or peak of a Psync signal or Psync values (P_{sync_peak}) is determined for an expiratory phase of a breathing cycle. At operation 624, a first time point for when the P_{sync_peak} occurs is identified and/or recorded. At operation 626, a Psync value for a threshold percentage (P_{sync_% thresh}) of P_{sync_peak} is determined. At operation 628, a second time point for when P_{sync_% thresh} occurs is identified and/or recorded. At operation 630, a first difference between P_{sync_peak} and P_{sync_% thresh} is determined and/or calculated. At operation 632, a second difference between the second time point and the first time point is determined. For example, a duration between the first time point and the second time point is determined. At operation 634, a quotient of the first difference and the second difference is determined. The determined quotient may be the rate of change of the Psync signal, such as the rate of change determined in operation 606 of method 600 depicted in FIG. 6A. The quotient may be obtained by dividing the first difference by the second difference. Accordingly, the quotient may have units of cmH₂O/s², or equivalent units.

FIG. 6D depicts an example method 640 for classifying respiratory mechanics. The example method 640 may be used in performing operation 608 of method 600 depicted in FIG. 6A. At operation 642, a rate of change of a Psync signal is compared to a first mechanics threshold (MT₁) to determine whether the rate of change is greater than the first mechanics threshold (MT₁). The rate of change may be the rate of change determined in method 620 depicted in FIG. 6C, and/or the rate of change may also be a P_{sync_slope} value. If, in operation 642, the rate of change is determined to not be greater than the first mechanics threshold (MT₁), then the respiratory mechanics of the patient are classified as obstructive in operation 644. If, in operation 642, the rate of change is determined to be greater than the first mechanics threshold (MT₁), then method 640 flows to operation 646.

At operation 646, rate of change of a Psync signal is compared to a second mechanics threshold (MT₂) to determine whether the rate of change is greater than the second mechanics threshold (MT₂). If, in operation 646, the rate of change is determined to not be greater than the second mechanics threshold (MT₂), then the respiratory mechanics of the patient are classified as normal in operation 648. If, in operation 646, the rate of change is determined to be greater than the second mechanics threshold (MT₂), then the respiratory mechanics of the patient are classified as restrictive in operation 650.

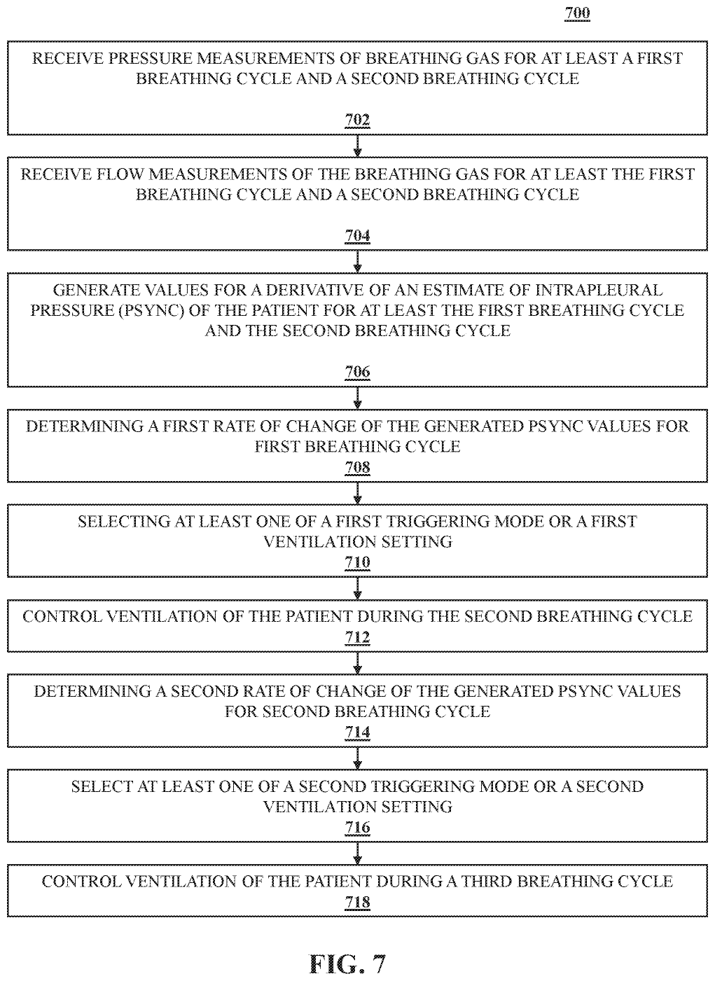

FIG. 7 depicts another example method 700 for providing mechanical ventilation based on detected respiratory mechanics. Method 700 is similar to method 600 depicted in FIG. 6A with the exception that method 700 explicitly includes multiple breathing cycles. At operation 702, pressure measurements of breathing gas provided by the ventilator are received for a first breathing cycle and a second breathing cycle. The second breathing cycle may be subsequent to the first breathing cycle. In some examples, the second breathing cycle may be immediately subsequent to the first breathing cycle, while in other examples there may be intervening breathing cycles in between the first breathing cycle and the second breathing cycle. The pressure measurements may include a Pᵢₙₛₚ value that is an inspiratory pressure transducer measurement and a Pₑₓₚ value that is an expiratory pressure transducer measurement. At operation 704, flow measurements of the breathing gas are received for the first breathing cycle and the second breathing cycle. The flow measurements may include a Qᵢₙₛₚ value that is an inspiratory flow sensor measurement and a Qₑₓₚ value that is an expiratory flow sensor measurement. The pressure measurements received in operation 704 and flow measurements received in operation 704 may be received for each sample period or computation cycle of the ventilator. Operations 702 and 704 may be performed concurrently or as part of the same operation.

At operation 706, values for a derivative of an estimate of intrapleural pressure (Psync) are generated for the first breathing cycle and the second breathing cycle based on the pressure measurements received in operation 702 and the flow measurements received in operation 704. At operation 708, a first rate of change for the generated Psync values during an expiratory phase of the first breathing cycle is determined. The first rate of change of the Psync values may be determined as described herein, such as by using Equation 1 provided above and/or the operations set forth in method 620 depicted in FIG. 6C. Based on the first rate of change, the respiratory mechanics of the patient may also be classified in operation 708.

At operation 710, based on the first rate of change and/or the classified respiratory mechanics determined in operation 708, at least one of a first triggering mode and/or a first setting of ventilation, such as a setting of a triggering mode or cycling mode, are determined. Operation 710 may be substantially the same as or substantially similar to operation 610 in method 600 depicted in FIG. 6A. At operation 712, ventilation of the patient for the second breathing cycle is controlled based on the first triggering mode and/or the first setting(s) for the triggering mode that were selected in operation 710.

At operation 714, a second rate of change for the generated Psync values during an expiratory phase of the second breathing cycle is determined. The second rate of change of the Psync values may be determined as described herein, such as by using Equation 1 provided above and/or the operations set forth in method 620 depicted in FIG. 6C. Based on the second rate of change, the respiratory mechanics of the patient may also be classified in operation 714.

At operation 716, based on the second rate of change and/or the classified respiratory mechanics determined in operation 708, at least one of a second triggering mode and/or a second setting of ventilation, such as a setting of a triggering mode or cycling mode, is determined. Operation 716 may be substantially the same as or substantially similar to operation 610 in method 600 depicted in FIG. 6A. At operation 718, ventilation of the patient for a third breathing cycle is controlled based on the second triggering mode and/or the second setting(s) for the triggering mode that were selected in operation 710.

Those skilled in the art will recognize that the methods and systems of the present disclosure may be implemented in many manners and as such are not to be limited by the foregoing exemplary aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein.

In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the present invention. Numerous other changes may be made which will readily suggest themselves to those skilled in the art and which are encompassed in the spirit of the disclosure and as defined in the claims.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with many systems such as ventilators for non-human patients, invasive or non-invasive ventilation, and other gas transport systems, and various types of event detection.

What is claimed is:

1. A mechanical ventilator for providing mechanical ventilation of a patient, the ventilator comprising:
a pressure sensor;
a flow sensor;
a processor; and
memory, storing instructions that when executed by the processor, cause the ventilator to perform a set of operations comprising:
receiving, from the pressure sensor, pressure measurements of breathing gas;
receiving, from the flow sensor, flow measurements of the breathing gas;
based on the pressure measurement and the flow measurement, generating a derivative of an estimate of intrapleural pressure (Psync signal) of the patient;
determining a rate of change of the Psync signal over a portion of an expiratory phase;
based on the determined rate of change, classifying respiratory mechanics as an obstructive respiratory condition;
altering a cycling sensitivity based on the obstructive classification; and
controlling ventilation of the patient based on the cycling sensitivity.

2. The ventilator of claim 1, wherein determining the rate of change of the Psync signal comprises:
identifying a maximum value of the Psync signal during the expiratory phase;
determining a Psync value for a threshold percentage of the identified maximum Psync value; and
wherein the determined rate of change is between the identified maximum Psync value and the determined Psync value for the threshold percentage.

3. The ventilator of claim 1, wherein altering the cycling sensitivity comprises altering a percentage sensitivity value.

4. The ventilator of claim 3, wherein controlling ventilation of the patient includes cycling, from an inspiratory phase to an expiratory phase, based on the percentage sensitivity value.

5. The ventilator of claim 3, wherein classifying the respiratory mechanics comprises comparing the determined rate of change to a mechanics threshold.

6. The ventilator of claim 5, wherein the mechanics threshold is 30 cmH₂O/s².

7. The ventilator of claim 5, wherein the obstructive classification is based on the rate of change being below the mechanics threshold.

8. The ventilator of claim 1, wherein the operations further comprise, based on the determined rate of change, selecting a triggering mode.

9. The ventilator of claim 8, wherein the selected triggering mode is one of flow triggering, pressure triggering, or signal distortion triggering.

10. A ventilator-implemented method for providing mechanical ventilation of a patient, the method comprising:
receiving pressure measurements of breathing gas;
receiving flow measurements of the breathing gas;
based on the pressure measurement and the flow measurement, generating values for a derivative of an estimate of intrapleural pressure (Psync) of the patient;
determining a rate of change of the generated Psync values over a portion of an expiratory phase of a breathing cycle;
based on the determined rate of change, selecting at least one of a triggering mode or a setting for ventilation; and
controlling ventilation of the patient based on the selected at least one of the triggering mode or the setting for ventilation.

11. The method of claim 10, wherein determining the rate of change of the Psync values comprises:
identifying a maximum of the Psync values during the expiratory phase;
determining a Psync value for a threshold percentage of the identified maximum Psync value; and
wherein the determined rate of change is between the identified maximum Psync value and the determined Psync value for the threshold percentage.

12. The method of claim 10, further comprising, based on the rate of change, classifying respiratory mechanics of the patient.

13. The method of claim 12, wherein the respiratory mechanics of the patient is one of normal, restrictive, or obstructive.

14. The method of claim 12, wherein classifying the respiratory mechanics comprises comparing the determined rate of change to a mechanics threshold.

15. The method of claim 14, where the mechanics threshold is 30 cmH₂O/s².

16. The method of claim 14, wherein the patient is classified as obstructive if the rate of change is below the mechanics threshold.

17. A ventilator-implemented method for providing mechanical ventilation of a patient, the method comprising:
receiving pressure measurements of breathing gas for at least a first breathing cycle and a second breathing cycle, the second breathing cycle occurring subsequent to the first breathing cycle;
receiving flow measurements of the breathing gas for at least the first breathing cycle and a second breathing cycle;
based on the pressure measurement and the flow measurement, generating values for a derivative of an estimate of intrapleural pressure (Psync) of the patient for at least the first breathing cycle and the second breathing cycle;
for an expiratory phase of the first breathing cycle, determining a first rate of change of the generated Psync values;
based on the determined first rate of change, selecting at least one of a first triggering mode or a first setting for ventilation;
controlling ventilation of the patient during the second breathing cycle based on the selected at least one of the first triggering mode or the first setting;
for an expiratory phase of the second breathing cycle, determining a second rate of change of the generated Psync values;
based on the determined second rate of change, selecting at least one of a second triggering mode or a second setting for ventilation; and
controlling ventilation of the patient during a third breathing cycle based on the selected at least one of the second triggering mode or the second setting for ventilation, wherein the third breathing cycle occurs after the second breathing cycle.

18. The method of claim 17, wherein determining the first rate of change comprises:
identifying a maximum of the Psync values during the expiratory phase of the first breathing cycle;
determining a Psync value, in the expiratory phase of the first breathing cycle, for a threshold percentage of the identified maximum Psync value; and
wherein the determined first rate of change is between the identified maximum Psync value and the determined Psync value for the threshold percentage.

19. The method of claim 17, further comprising, based on the second rate of change, classifying respiratory mechanics of the patient.

20. The method of claim 19, wherein classifying the respiratory mechanics comprises comparing the determined rate of change to a mechanics threshold.
